(19) United States
(12) Patent Application Publication
(10) Pub. No.: US 2020/0330416 A1
(43) Pub. Date: Oct. 22, 2020

(54) METHOD AND COMPOSITION FOR IMPROVED HYDRATION IN ANIMALS

(71) Applicants: Alon ROSENBERG, Rehovot (IL); KALMARNA LIMITED, Road Town (VG)

(72) Inventors: Alon ROSENBERG, Rehovot (IL); Abraham MILSTEIN, Rehovot (IL); Ava Marie FIRTH, West Bridgford (GB); Simon VAN DALSEM, Alkmaar (NL); Arie HALPERN, Pully (CH)

(21) Appl. No.: 16/865,697

(22) Filed: May 4, 2020

Related U.S. Application Data
(63) Continuation-in-part of application No. PCT/IL2019/050472, filed on Apr. 29, 2019.
(60) Provisional application No. 62/664,174, filed on Apr. 29, 2018.
(60) Provisional application No. 62/723,496, filed on Aug. 28, 2018.

(51) Int. Cl. A61K 31/198; A61K 31/194; A61K 9/06; A61K 9/08; A61K 9/00; A61K 45/06; A61K 31/704

(52) U.S. Cl. CPC A61K 31/198; A61K 31/194; A61K 9/06; A61K 31/704; A61K 9/0095; A61K 9/0056; A61K 45/06; A61K 9/08

(57) ABSTRACT

The present invention discloses oral isotonic compositions to provide hydration to mammals in need, characterized by no residual chlorine taste, comprising at least three members of a group consisting of steviol glycoside, citric acid monohydrate, glutamic acid, monosodium glutamate and glycine.

### CROSS REFERENCE TO RELATED APPLICATIONS

This application is a Continuation-in-Part of PCT International Application No. PCT/IL2019/050471, filed Apr. 29, 2019, which claims priority from provisional application Nos. 62/664,174 filed on Apr. 29, 2018 and 62/723,496 filed on Aug. 28, 2018. All of these applications are hereby incorporated by reference in their entirety.

### FIELD OF THE INVENTION

This invention relates to a method for increasing hydration in animals, and more particularly, in cats and dogs.

### BACKGROUND OF THE INVENTION

Physical activity in hot environments can increase the risk of heat stress or heat stroke in pets. Pets, or companion animals, are affected by high temperatures and humidity; thus, heat tolerance is influenced by acclimatization to the environment, physical fitness, and hydration state.

Since dogs have minimal sweating capacity, thermoregulation relies primarily on evaporative mechanisms through panting. Given dogs' inability to sweat (except through the pads of their feet), dogs rely on panting to cool them down and high temperatures, coupled with a lack of water, can severely interfere with this process, leading to dehydration. High humidity can also negatively affect a canine's capacity for evaporative cooling. Even in moderate temperatures, dogs can even suffer from dehydration; see Sodhi, Nidhi. “Dog hydration strategies.” Aust Vet J 2018; 96(3):N20 (2018), incorporated herein as a reference.

In addition to the environmental factors of heat and humidity, the dog's adaptation to environmental conditions (acclimatization), overall fitness/conditioning and state of hydration are thought to be major factors that impact heat tolerance. Canine thermoregulation involves the inhalation of cooler, dryer air through the nose and mouth, which causes evaporative heat loss from the nasal and oral mucosa and tongue, and the exhalation of hotter, moister air. In conditions in which the rate of heat generation is greater than the rate of heat dissipation, increased salivation and lingual blood flow allow for convection and evaporative cooling). Evaporative cooling results in electrolyte-free water loss (estimated at ˜10 mL/kg/h) which can contribute to dehydration, see Otto, Cynthia M., et al. “evaluation of three hydration strategies in Detection Dogs Working in a hot environment.” Frontiers in veterinary science 4 (2017), incorporated herein as a reference.

Dogs primarily cool themselves through panting, rather than sweating through skin surfaces. The only areas of skin that a dog uses for sweating are its nose and footpads. Panting necessarily involves the exhalation of warm air from the lungs. This air is also moist, and therefore panting may result in considerable water losses from the dog. Dogs may become measurably dehydrated after only 15 minutes of exercise, losing an average of 0.83% of their body weight and showing visually detectable changes in skin turgor (Goucher T. Amer. J. Vet. Research 2019 80(2):123-8, incorporated herein as a reference). Panting is an inefficient method of cooling however, and dogs may become hyperthermic after only a short period of exercise. This was demonstrated in the study by Zanghi, in which dogs had an increase in core body temperatures of 2.5-3° C. after only 30 minutes of exercise in moderate environmental temperatures (Zanghi et al., Front. Vet. Sci. 2018, 5:202, incorporated herein as a reference). This is a dangerous rise in body temperature. In the study by Evans, heat stroke was the most common cause of medical (non-behavioral) discharge in military working dogs less than 5 years old, accounting for 8.2% of the discharges (Evans R. I., J. Amer. Vet. Med. Assoc, 2007, 231(8):1215-20, incorporated herein as a reference). Studies show that well-hydrated dogs are less prone to dehydration following physical activity (Baker et al., J. Physiol. Pharmacol. 1991, 42(3):305-316, incorporated herein as a reference). Oral rehydration solutions have also been shown to be more effective than either plain water or subcutaneous fluids in minimizing markers of dehydration such as increased creatinine, increased hematocrit, increased total plasma protein and decreased total CO₂ (Otto, C. M., Front. Vet. Sci. 2013 4(October) 174, incorporated herein as a reference).

Dehydration in horses is a major concern for those involved in the equine industry, especially during the hot summer months. At these times high temperatures can induce increased sweating rates in horses, potentially causing water and electrolyte losses to reach dangerous levels, see Green, B. L., and J. L. Wahrmund. “143 Impact of Hydration Supplements on Blood Electrolyte Concentrations of Exercised Horses. During the Summer.” Journal of Animal Science94.suppl_1 (2016): 69-70, incorporated herein as a reference.

The domestic cat is adapted to retain water by producing urine which is very concentrated compared to most other mammals. Producing highly concentrated urine, however, can have deleterious effects, such as enhancing development of urinary stones and other less well-defined urinary tract conditions such feline idiopathic cystitis. Manipulating hydration is the most appealing approach to prevent dehydration and increase heat tolerance and workability. Prior documents disclose methods and hydration strategies for pets: Palatability enhancers for pet food are known in the art. U.S. Pat. Nos. 6,354,920 and 6,350,485 to Brunner, incorporated herein as a reference, disclose the use of tetrasodium pyrophosphate as a palatability enhancer for dry and semi-moist cat food. U.S. Pat. No. 5,186,964 to Gierhart et al. discloses the use of a pyrophosphate salt or an acid phosphate salt as a palatability enhancer for dry pet food. U.S. Pat. No. 7,244,460 to Lee et al. discloses the use of a tripolyphosphate salt as a palatability enhancer for dry and semi-dry pet food. The use of a palatability enhancer as a flavoured drinking water additive is discloses in US patent application 20140127352 to Woodward et al. US patent application 20140127352 to Woodward et al discloses flavoured drinking water additives and flavoured drinking water compositions, as well as methods for increasing water consumption by an animal and improving hydration for animals using the flavoured drinking water additives and flavoured drinking water compositions of Woodward invention based on pyrophosphate salts. US patent application 20180085309 to Baskins incorporated herein as a reference discloses a pet hydration System, comprising of specific ranges of vitamins and minerals conducive to a pet's health, with such nutrients placed into a liquid beverage and ultimately dispensed via a compartmentalized beverage dispenser. The compartmentalized beverage dispenser is formed such that separate liquids are measured out and dispensed to the pet in a manner that the pet receives the optimal amount of nutrition. US patent application US20170172180 to Znaghi discloses composition and methods for improving hydration and water intake in an animal. In one embodiment, a hydration composition can comprise water and a hydration additive, where the hydration additive consists essentially of a sugar alcohol and a protein, where the sugar alcohol includes glycerol and the protein includes whey. Additionally, a method of improving hydration and water intake in an animal can comprise administering the hydration composition with water to the animal. U.S. Pat. No. 9,884,035B2 to Brockman et al., discloses methods and compositions for improving the level of hydration in a cat. Cats fed diets containing certain amounts and ratios of arachidonic acid and eicosapentaenoic acid will be sufficiently hydrated and at reduced risk for development of urinary stones, feline idiopathic cystitis.

Cats commonly experience complications of dehydration also, but it is more of a chronic problem. Cats are notoriously fussy drinkers and this fact is recognized by the International Cat Care Association and World Small Animal Veterinary Association (https://icatcare.org/advice/how-guides/how-encourage-your-cat-drink).

Because of these habits, domestic cats are quite susceptible to chronic dehydration which results in the production of highly concentrated urine. Chronic dehydration may lead to renal disease, which is a common cause of illness in cats. Producing highly concentrated urine, in turn, can have further deleterious effects, such as lower urinary tract disease (LUTD), which is also common in cats. There is evidence that increasing fluid intake and urine dilution may reduce the recurrence of LUTD (Grant, D. J. Feline Med. Surg. 2010 12(6): 431-4, incorporated herein as a reference).

Mink have been farmed for fur in the United States for 130 years. In 2010, the U.S. ranked fifth in production behind Denmark, China, the Netherlands, and Poland. Mink typically breed in March and give birth to their litters in May. (see Wikipedia, a “fur faming”, incorporated herein as a reference).

Mortality in mink kits held under conventional farm conditions is believed to be related to dehydration, see Brink, A-L., Leif Lau Jeppesen, and Knud Erik Heller. “Behaviour in suckling mink kits under farm conditions: effects of accessibility of drinking water.” Applied Animal Behaviour Science 89.1 (2004): 131-137; incorporated herein as a reference.

Maintaining adequate hydration levels is vital for health in animals. However, ensuring adequate water intake in some companion animals, particularly cats and dogs, can be quite challenging in some situations. Lack of adequate water intake can lead to dehydration, heat stroke and renal/urinary disease.

Thus, increasing water intake, is a major factor in reducing or preventing dehydration in companion animals. However, dogs and cats are not easily enticed to increase their water intake. In order to increase drinking in cats and dogs, palatants and flavorings have been added to drinking water provided to pets. WO 2009/009879 to Ramsey et al, discloses a beverage composition for promoting hydration in animals, comprising water, an attractant having a flavor and odor reminiscent of meat and a biologically active enzyme or plant extract. US 2011/02668664 to Martinez et al, discloses and effervescent composition for increasing water intake in animals, comprising an effervescent agent, and a meat, fish or dairy based palatant. An additional composition for increasing water intake in animals is disclosed by Zanghi, in US 2017/0172180. The composition comprises water, glycerol and whey protein. US 2008/009879 to Gaggioti, discloses a drink for animals based on water containing meat or meat extracts, vegetable extracts, minerals and vitamins.

As shown above, physical activity is known to increase an individual's water requirement, particularly while exercising. Additionally, stress related to travel, boarding, or illness also compromises hydration status in cats, minks and dogs and horses. Thus, there remains a need for compositions and methods to improve water intake and increase hydration in companion animals such as dogs, cats and horses.

### SUMMARY OF THE INVENTION

It is one object of the invention to disclose an oral isotonic compositions characterized by no residual chlorine taste for the hydration of mammals comprising at least three members of a group consisting of steviol glycoside, citric acid monohydrate, monosodium glutamate, glycine, mixtures and derivatives thereof.

Chlorinous flavours at the food and drinks are the leading cause of human and animals' complaints, dissatisfaction and dehydration, see e.g., Piriou, Philippe, et al. “Chlorinous flavour perception in drinking water.” Water Science and Technology 49.9 (2004): 321-328. A chlorine-free taste in foods and drinks are hence a long-felt need.

It is another object of the invention to disclose an oral isotonic compositions characterized by no residual chlorine taste for the hydration of mammals comprising (a) at least three members of a group consisting of steviol glycoside, citric acid monohydrate, monosodium glutamate, glycine, mixtures and derivatives thereof; and (b) prebiotic any derivatives and mixtures thereof.

Prebiotics are food ingredients that induce the growth or activity of beneficial microorganisms (e.g., bacteria and fungi). The term ‘prebiotic’ hence refers hereinafter, inter alia and in a non-limiting manner, to both of endogenous and exogenous sources-driven prebiotics, and to at least one member of a group consisting of edibles being non-digestible and resistant to breakdown by stomach acid and enzymes in the human gastrointestinal tract; edibles selectively fermented by intestinal microorganisms; and edibles selectively target and stimulate the growth and activity of beneficial bacteria; see Hutkins R W et al., (2016). “Prebiotics: why definitions matter”. Curr Opin Biotechnol. 37: 1-7. The term also refers to synbiotics, e.g., food ingredients or dietary supplements combining probiotics and prebiotics in a form of synergism, see DeVrese M, Schrezenmeir J (2008) Probiotics, prebiotics, and synbiotics in food biotechnology (pp. 1-66). Springer Berlin Heidelberg which is incorporated herein as a reference.

It is another object of the invention to disclose an isotonic drink characterized by no residual chlorine taste for the hydration of mammals comprising at least three members of a group consisting of steviol glycoside, citric acid monohydrate, monosodium glutamate, glycine, mixtures and derivatives thereof and any derivatives and mixtures thereof.

It is another object of the invention to disclose an isotonic drink compositions characterized by no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an apple flavoured horses' isotonic drink characterized by no residual chlorine taste comprising (a) at least three members of a group consisting steviol glycoside, citric acid monohydrate, monosodium glutamate, glycine, mixtures and derivatives thereof; and (b) an apple-flavouring agent.

The term ‘apple-flavouring agent’ refers herein after, inter alia and in a non-limiting manner, to one or more of the following: apple extracts, apple flavour No. 64625, malic acids, esters, salts and derivatives thereof, apple flavour fragrance, e.g., as defined in CN patent applications 107279615, 104830607 and 105212170, JP patent application 2011152095, Maxarome AC 1632, all are incorporated herein as references, taste enhancers, emulsifiers and stabilizers thereof.

Maxarome AC 1632 is a concentrated, high performance flavour developed specifically for inclusion into animal feeds where it will improve palatability and encourage feed intake. Maxarome AC 1632 includes Vanillin and other aldehydes, together with mixed esters. Maxarome AC 1632 has the following characteristics: Physical Appearance: Beige-pale brown free flowing powder; Volatile Matter: 38.94%; Solubility: Insoluble; Bulk Density: 0.625 g/cm3; Flavour Classification: Nature Identical; Flavour Type: Spicy Apple; Product Classification: Conforms to EU Regulation 1831/2003—Annex 1, 2(b); Pre-mixture of flavouring compounds; Added Colouring: None Added; Antioxidants: None Added.

It is another object of the invention to disclose an apple flavoured horses' isotonic drink characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.

It is another object of the invention to disclose an isotonic taste enhancer, e.g., fluid, solid or gel-formed taste enhancer, edible meal topper, a treat tec., characterized by no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic taste enhancer characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an isotonic taste enhancer compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic.

It is another object of the invention to disclose an isotonic apple flavoured horses' taste enhancer characterized by no residual chlorine taste comprising (a) at least three members of a group consisting steviol glycoside, citric acid monohydrate, monosodium glutamate, glycine, mixtures and derivatives thereof; and (b) an apple-flavouring agent.

It is another object of the invention to disclose an isotonic Apple flavoured horses' taste enhancer characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.

It is another object of the invention to disclose an edible powder characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an edible powder characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste comprising (a) at least three members of a group consisting steviol glycoside, citric acid monohydrate, monosodium glutamate, glycine, mixtures and derivatives thereof; and (b) an apple-flavouring agent.

It is another object of the invention to disclose an apple flavoured horses' edible powder characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.

It is another object of the invention to disclose an edible gel characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible gel compositions characterized by no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an edible isotonic gel characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an apple flavoured horses' edible isotonic gel characterized by no residual chlorine taste comprising (a) at least three members of a group consisting steviol glycoside, citric acid monohydrate, monosodium glutamate, glycine, mixtures and derivatives thereof; and (b) an apple-flavouring agent.

It is another object of the invention to disclose an apple flavoured horses' edible isotonic gel characterized by no residual chlorine taste comprising (a) at least three members of a group consisting steviol glycoside, citric acid monohydrate, monosodium glutamate, glycine, mixtures and derivatives thereof (b) an apple-flavouring agent; and (c) prebiotic.

It is another object of the invention to disclose a solid shear thinning thixotropic edible gel characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose a solid shear thinning thixotropic edible gel characterized by no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof and (b) an apple-flavouring agent.

It is another object of the invention to disclose an apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof (b) an apple-flavouring agent; and (c) prebiotic.

It is another object of the invention to disclose an oral isotonic compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof and (c) prebiotic any derivatives and mixtures thereof.

It is another object of the invention to disclose an isotonic drink characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) an apple-flavouring agent.

It is another object of the invention to disclose an apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an edible powder compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof and (c) prebiotic.

It is another object of the invention to disclose an apple flavoured horses' edible powder characterized by no residual chlorine taste comprising (a) a member of a group consisting of vaccines, iron-containing compositions, minerals, salts, amino, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is another object of the invention to disclose an apple flavoured horses' edible powder characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an ral isotonic compositions characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an oral isotonic compositions characterized by chicken flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose isotonic drink characterized by chicken flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic taste enhancer characterized by chicken flavour and no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible powder characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by chicken flavour and no residual chlorine taste for the hydration of mammals comprising at least three members of a group consisting (a) steviol glycoside, citric acid monohydrate, monosodium glutamate, glycine, mixtures and derivatives thereof and (b) prebiotic any derivatives and mixtures thereof.

It is another object of the invention to disclose an edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible gel compositions characterized by chicken flavour and no residual chlorine taste for the hydration of mammals comprising at least three members of a group consisting (a) steviol glycoside, citric acid monohydrate, monosodium glutamate, glycine, mixtures and derivatives thereof and (b) prebiotic any derivatives and mixtures thereof.

It is another object of the invention to disclose an edible isotonic gel characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by chicken flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose a solid shear thinning thixotropic edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose a solid shear thinning thixotropic edible gel characterized by chicken flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an oral isotonic compositions characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an oral isotonic compositions characterized by fish flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by fish flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic taste enhancer characterized by fish flavour and no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible powder characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible gel characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an Edible gel compositions characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible isotonic gel characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by fish flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an solid shear thinning thixotropic edible gel characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an solid shear thinning thixotropic edible gel characterized by fish flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an oral isotonic compositions characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an oral isotonic compositions characterized by beef flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an isotonic drink characterized by beef flavour and no residual chlorine taste for the hydration of mammals comprising at least three members of a group consisting of steviol glycoside, citric acid monohydrate, monosodium glutamate, glycine, mixtures and derivatives thereof and any derivatives and mixtures thereof.

It is another object of the invention to disclose an isotonic drink compositions characterized by beef flavour and no residual chlorine taste for the hydration of mammals comprising at least three members of a group consisting (a) steviol glycoside, citric acid monohydrate, monosodium glutamate, glycine, mixtures and derivatives thereof and (b) prebiotic any derivatives and mixtures thereof.

It is another object of the invention to disclose an isotonic taste enhancer characterized by beef flavour and no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible powder characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible gel characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible gel compositions characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible isotonic gel characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by beef flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose a solid shear thinning thixotropic edible gel characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose a solid shear thinning thixotropic edible gel characterized by beef flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof

It is still another object of the invention to disclose a method selected from a group consisting of enhancing palatability, fast hydrating, gut activity supporting, providing micro-nutrition supplementation, and enhancing digestion and absorption; t; wherein said method comprising step of administrating a mammal a composition as defined in any of the above.

It is an object of this invention to provide a method of inducing an animal to increase its water intake, comprising providing to said animal a water composition comprising a meat or chicken hydrolysate, meat or chicken flavorings, at least one sweetener, and at least one component selected from glutamic acid or monosodium glutamate.

It is another object of this invention to provide a method of producing a solution for inducing an animal to increase its water intake comprising mixing a meat or chicken hydrolysate, meat or chicken flavorings, water, at least one sweetener, and at least one component selected from glutamic acid or monosodium glutamate.

It is a further object of this invention to provide a method of inducing an animal to increase its' water intake, wherein said animals are cats and dogs.

### BRIEF DESCRIPTION OF THE FIGURES

The present invention will be readily understood by the following detailed description, in conjunction with the accompanying drawings and in which:

FIG. 1: depicting a non-limiting example of a preferred gel embodiment of the current invention.

FIG. 2: depicting a non-limiting example of a preferred gel embodiment of the current invention.

FIG. 3: shows the average cat consumption of a composition for cats disclosed in the present application vs. consumption of water

FIG. 4: shows results of initial palatability test (“first choice”) for cats offered water and a composition for cats disclosed in the present application.

FIG. 5: shows results of initial palatability test (“first choice”) for dogs offered water and a composition for dogs disclosed in the present application.

FIG. 6: shows the average dog consumption of a composition for dogs disclosed in the present application vs. consumption of water.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide compositions and methods for improving hydration in companion animals, especially cats and dogs. Specifically, the present invention discloses palatable oral formulations for the increase of water uptake by companion animals.

As used herein after, the terms “pet” or “companion animal” interchangeably refers hereinafter to an animal kept primarily for a person's company, protection, or entertainment rather than as a working animal, livestock, or laboratory animal. Popular pets are often noted for their attractive appearances, intelligence, and relatable personalities. Two of the most popular pets are dogs and cats. Other animals commonly kept include: pigs, ferrets, rabbits; rodents, such as gerbils, hamsters, chinchillas, rats, and guinea pigs; avian pets, such as parrots, passerines, and fowl; reptile pets, such as turtles, lizards and snakes; aquatic pets, such as fish, freshwater and saltwater snails, and frogs; and arthropod pets, such as tarantulas and hermit crabs.

As used hereinafter, the term “steviol” generally refers to steviol glycoside, Rebaudioside A extract.

The present invention discloses compositions for improving hydration in animals, and an associated method of providing said compositions to cats or dogs. These compositions are water based, and contain additives palatable to cats or dogs, thereby increasing water intake. The palatable additives comprise meat/chicken hydrolysates and/or flavoring, a sweet flavor, and glutamic acid (umami flavor).

The water used in the compositions of the present invention is demineralized water, since cats and dogs prefer not to drink tap water containing chlorine.

In one embodiment, a composition for increasing fluid intake in cats comprises water, glucose, sodium chloride, potassium chloride, glycine, liquid liver hydrolysate, glutamic acid or monosodium glutamate, and taurine.

In another other embodiment, the composition for increasing fluid intake in cats further comprises at least one additional component, selected from the group consisting of xanthan gum, monosodium phosphate, meat or chicken flavouring, and fructo-oligosaccharide.

In another embodiment, a composition for increasing fluid intake in dogs comprises: water, glucose, sodium chloride, potassium chloride, glycine, liquid liver hydrolysate, meat or chicken flavouring, and glutamic acid or monosodium glutamate.

In another embodiment, the composition for increasing fluid intake in dogs further comprises at least one additional component, selected from the group consisting of monosodium phosphate, fructo-oligosaccharide, xanthan gum, and rebaudioside A.

In another embodiment, the composition for increasing fluid intake in dogs further comprises burnt sugar syrup.

The current invention discloses oral chlorine-free compositions for hydration of humans and animals, characterized by enhanced palatability, fast hydration, gut support activity, micro-nutrition supplementation, and enlaced digestion and absorption. It is in the scope of the invention wherein aforesaid non-genetically-modified compositions, products and formulations comprises or based on commercially available Px (trademark) by Tonisity International Ltd, namely, a composition, e.g., defined below:

TABLE 1
Ingredients of Px formulation.

| Ingredients | (%) |
|---|---|
| glucose anhydrous | 1.5540 |
| monosodium glutamate | 0.3900 |
| sodium chloride, vacuum dried | 0.2201 |
| citric acid anhydrous | 0.1860 |
| potassium chloride | 0.1500 |
| glycine | 0.1500 |
| hydrolyzed whey protein isolate | 0.1500 |
| monosodium dihydrogen phosphate | 0.1000 |
| xanthan gum | 0.0500 |
| 1-glutamic acid | 0.0400 |
| stevia (min. 85% steviol glycoside, Rebaudioside A extract) | 0.0100 |
| Total ingredients | 3.0000 |
| demineralized water | 97.000 |

The composition consists at least two members of a group consisting of steviol glycoside, citric acid monohydrate, monosodium glutamate and glycine, this composition characterized by that that the composition consists at least three members of a group consisting of steviol glycoside, citric acid monohydrate, monosodium glutamate and glycine. Additionally, or alternatively, this composition is characterized by that that said composition consisting of steviol glycoside, citric acid monohydrate, monosodium glutamate and glycine.

Additionally, or alternatively, the composition of the current invention also discloses a flavouring agent selected form a group consisting of apple, chicken, beef or fish flavours, with or without prebiotics, and any derivatives and mixtures thereof.

Additionally, or alternatively, the composition of the current invention is a palatable carrier of at least one compound of a group consisting of vaccines, iron-containing compositions, minerals, salts, amino acids, medicaments, vitamins, nutraceuticals, antibiotics and food supplements, for nourishing and treating mammals. Thus, the composition of the current invention overcomes the risks of needle administration of the compounds, decreases labor and manpower needed for injecting the treated mammals, and increasing compliance to treatment.

The current invention discloses compositions comprising isotonic drinks (ready-to-use liquids), isotonic edible powders, isotonic taste enhancer, e.g., fluid, edible solid or gel-formed taste enhancer, edible meal toppers (“Gravy”), a treat etc., characterized by no residual chlorine taste for the hydration of mammals, such as cats, dogs, horses, and mink.

These compositions of the current invention compositions comprise:

Equine formulations: formulations for feeding horses of all type of services: horses for work, sport entrainment, culture, warfare, and for therapeutic uses.

These equine formulations comprise e.g., one of the followings:
a. An apple flavoured horses' isotonic drink characterized by no residual chlorine taste comprising (a) at least three members of a group consisting steviol glycoside, citric acid monohydrate, monosodium glutamate, glycine, mixtures and derivatives thereof; and (b) an apple-flavouring agent.
b. An apple flavoured horses' isotonic drink characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.
c. An isotonic apple flavoured horses' taste enhancer characterized by no residual chlorine taste comprising (a) at least three members of a group consisting steviol glycoside, citric acid monohydrate, monosodium glutamate, glycine, mixtures and derivatives thereof; and (b) an apple-flavouring agent.
d. An isotonic Apple flavoured horses' taste enhancer characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.
e. An apple flavoured horses' edible powder characterized by no residual chlorine taste comprising (a) at least three members of a group consisting steviol glycoside, citric acid monohydrate, monosodium glutamate, glycine, mixtures and derivatives thereof; and (b) an apple-flavouring agent.
f. An apple flavoured horses' edible powder characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.
g. An apple flavoured horses' edible isotonic gel characterized by no residual chlorine taste comprising (a) at least three members of a group consisting steviol glycoside, citric acid monohydrate, monosodium glutamate, glycine, mixtures and derivatives thereof; and (b) an apple-flavouring agent.
h. An apple flavoured horses' edible isotonic gel characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.
i. An apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof; and (b) an apple-flavouring agent.
j. An apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.
k. An apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) an apple-flavouring agent.
l. An apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.
m. An apple flavoured horses' edible powder characterized by no residual chlorine taste comprising (a) a member of a group consisting of vaccines, iron-containing compositions, minerals, salts, amino, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.
n. An apple flavoured horses' edible powder characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) an apple-flavouring agent; and (d) prebiotic.

A non-limiting example for horse formulation is Px-E version 3, see Table 2.

Topper formulations: taste enhancers, formulations for enhancing palatably of commercially available dry food for pets.

These topper formulations comprise:
a. An isotonic taste enhancer, e.g., fluid, solid or gel-formed taste enhancer, edible meal topper, a treat tec., characterized by no residual chlorine taste for the hydration of mammals comprising at least three members of a group consisting steviol glycoside, citric acid monohydrate, monosodium glutamate, glycine, mixtures and derivatives thereof.
b. An isotonic taste enhancer characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. An isotonic taste enhancer characterized by no residual chlorine taste for the hydration of mammals comprising at least three members of a group consisting of steviol glycoside, citric acid monohydrate, monosodium glutamate, glycine, mixtures and derivatives thereof.
d. An isotonic taste enhancer composition characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic.
e. An isotonic taste enhancer characterized by chicken flavour and no residual chlorine taste for the hydration of mammals comprising at least three members of a group consisting steviol glycoside, citric acid monohydrate, monosodium glutamate, glycine, mixtures and derivatives thereof.
f. An isotonic taste enhancer characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
g. An isotonic taste enhancer characterized by fish flavour and no residual chlorine taste for the hydration of mammals comprising at least three members of a group consisting steviol glycoside, citric acid monohydrate, monosodium glutamate, glycine, mixtures and derivatives thereof.
h. An isotonic taste enhancer characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
i. An isotonic taste enhancer characterized by beef flavour and no residual chlorine taste for the hydration of mammals comprising at least three members of a group consisting steviol glycoside, citric acid monohydrate, monosodium glutamate, glycine, mixtures and derivatives thereof.
j. An isotonic taste enhancer characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

A non-limiting example for a topper formulation is XDG version 2 or version 3, see Table 3.

Ready-to-use liquid formulations: non-chlorine isotonic drink formulations.

These ready-to-use drink formulations comprise:
a. An isotonic drink characterized by no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
b. An isotonic drink composition characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. An isotonic drink characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.
d. An isotonic drink compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.
e. An isotonic drink characterized by chicken flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
f. An isotonic drink composition characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
g. An isotonic drink characterized by fish flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
h. An isotonic drink composition characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
i. An isotonic drink characterized by beef flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
j. An isotonic drink composition characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

A non-limiting example for a ready-to-use liquid for dogs is X-D liver version 3, see Table 4. A non-limiting example for a ready-to-use liquid is an isotonic liquid for cats version 1, see Table 7.

Powder formulations: fast hydration palatable oral edible formulations.

These powder edible formulations comprise:
a. An edible powder characterized by no residual chlorine taste for the hydration of mammals comprising at least three members of a group consisting of steviol glycoside, citric acid monohydrate, monosodium glutamate, glycine, mixtures and derivatives thereof
b. An edible powder characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. An edible powder characterized by no residual chlorine taste for the hydration of mammals comprising at least three members of a group consisting of steviol glycoside, citric acid monohydrate, monosodium glutamate, glycine, mixtures and derivatives thereof
d. An edible powder composition characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
e. An edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.
f. An edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.
g. An edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof
h. An edible powder compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.
i. An edible powder characterized by chicken flavour and no residual chlorine taste for the hydration of mammals comprising at least three members of a group consisting of steviol glycoside, citric acid monohydrate, monosodium glutamate, glycine, mixtures and derivatives thereof.
j. An edible powder characterized by chicken flavour and no residual chlorine taste for the hydration of mammals comprising at least three members of a group consisting (a) steviol glycoside, citric acid monohydrate, monosodium glutamate, glycine, mixtures and derivatives thereof and (b) prebiotic any derivatives and mixtures thereof.
k. An edible powder characterized by fish flavour and no residual chlorine taste for the hydration of mammals comprising at least three members of a group consisting of steviol glycoside, citric acid monohydrate, monosodium glutamate, glycine, mixtures and derivatives thereof.
l. An edible powder characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
m. An edible powder characterized by beef flavour and no residual chlorine taste for the hydration of mammals comprising at least three members of a group consisting of steviol glycoside, citric acid monohydrate, monosodium glutamate, glycine, mixtures and derivatives thereof.
n. An edible powder characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

A non-limiting example for a powder suitable for dogs is X54 (see Table 5). A non-limiting example for a powder suitable for cats is isotonic powder mix for cats version 1, see Table 8.

Gel formulations: edible isotonic gel formulations, including inter alia firm gel or shear-thinning thixotropic edible gel.

These edible gel formulations comprise:
a. an edible gel characterized by no residual chlorine taste for the hydration of mammals comprising at least three members of a group consisting of steviol glycoside, citric acid monohydrate, monosodium glutamate, glycine, mixtures and derivatives thereof.
b. an edible gel composition characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. an edible isotonic gel characterized by no residual chlorine taste for the hydration of mammals comprising at least three members of a group consisting of steviol glycoside, citric acid monohydrate, monosodium glutamate, glycine, mixtures and derivatives thereof
d. an edible isotonic gel composition characterized by no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
e. a solid shear thinning thixotropic edible gel characterized by no residual chlorine taste for the hydration of mammals comprising at least three members of a group consisting of steviol glycoside, citric acid monohydrate, monosodium glutamate, glycine, mixtures and derivatives thereof
f. a solid shear thinning thixotropic edible gel characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
g. an edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof
h. an edible gel composition characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
i. an edible isotonic gel characterized by chicken flavour and no residual chlorine taste for the hydration of mammals comprising at least three members of a group consisting of steviol glycoside, citric acid monohydrate, monosodium glutamate, glycine, mixtures and derivatives thereof
j. an edible isotonic gel composition characterized by chicken flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
k. a solid shear thinning thixotropic edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof.
l. a solid shear thinning thixotropic edible gel characterized by chicken flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
m. an edible gel characterized by fish flavour and no residual chlorine taste for the hydration of mammals comprising at least three members of a group consisting of steviol glycoside, citric acid monohydrate, monosodium glutamate, glycine, mixtures and derivatives thereof
n. an edible gel composition characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
o. an edible isotonic gel characterized by fish flavour and no residual chlorine taste for the hydration of mammals comprising at least three members of a group consisting of steviol glycoside, citric acid monohydrate, monosodium glutamate, glycine, mixtures and derivatives thereof
p. an edible isotonic gel composition characterized by fish flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
q. a solid shear thinning thixotropic edible gel characterized by fish flavour and no residual chlorine taste for the hydration of mammals comprising at least three members of a group consisting of steviol glycoside, citric acid monohydrate, monosodium glutamate, glycine, mixtures and derivatives thereof.
r. a solid shear thinning thixotropic edible gel characterized by fish flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
s. an edible gel characterized by beef flavour and no residual chlorine taste for the hydration of mammals comprising at least three members of a group consisting of steviol glycoside, citric acid monohydrate, monosodium glutamate, glycine, mixtures and derivatives thereof
t. an edible gel composition characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
u. an edible isotonic gel characterized by beef flavour and no residual chlorine taste for the hydration of mammals comprising at least three members of a group consisting of steviol glycoside, citric acid monohydrate, monosodium glutamate, glycine, mixtures and derivatives thereof
v. an edible isotonic gel composition characterized by beef flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
w. a solid shear thinning thixotropic edible gel characterized by beef flavour and no residual chlorine taste for the hydration of mammals comprising at least three members of a group consisting of steviol glycoside, citric acid monohydrate, monosodium glutamate, glycine, mixtures and derivatives thereof.
x. a solid shear thinning thixotropic edible gel characterized by beef flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof

A non-limiting example for an edible gel is firm-gel carrageenan—LBG, see Table 6.

### Example 1

Tonisity Equine-Px-E version 3, see Table 2.

Problem: Horses that are subjected to long bouts of exercise will lose massive amounts of water and electrolytes. If the horse is not quickly rehydrated, this can become a serious emergency and lead to kidney failure.

Delivery: Powder

Properties: White color (closely resembles Tonisity Px); Pleasant apple aroma and flavouring; and Oral rehydration formula.

Uses: Dry top dressing sprinkled on feed; Mixed with water to create an isotonic solution; Mixed with other feeds (e.g., bran, beet pulp, etc.) to make a mash

Results: In trials, the mash and top dressing were very well received

Trials indicated that horses had better exercise tolerance, improved stamina and demeanor, and generally more energy.

Intended Uses: For working horses performing heavy exercise; after 3-day events; dressage horses; and Long-distance riding and racing.

Package Size Suggestions: 155 g scoop of the powder per horse per day, so package could be 1 or 2.5 kg.

TABLE 2
The ingredients of a preferred embodiment for
a horse nourishing formulation, Px-E version 3
Px-E Version 3

| | Solution | Dry Mix |
|---|---|---|
| Ingredients | % | % |
| Demineralised Water | 97.000 | |
| Glucose Anhydrous | 1.674 | 55.800 |
| Sodium Chloride | 0.220 | 7.334 |
| Potassium Chloride | 0.150 | 5.000 |
| Glycine | 0.150 | 5.000 |
| Xanthan Gum | 0.050 | 1.667 |
| Monosodium Phosphate | 0.100 | 3.333 |
| Citric Acid Anhydrous | 0.060 | 2.000 |
| Hydrolysed Whey Protein Isolate | 0.150 | 5.000 |
| L-Glutamic Acid | 0.040 | 1.333 |
| Monosodium Glutamate | 0.390 | 13.000 |
| Rebaudioside A 98% Min. | 0.010 | 0.333 |
| Thaumatin | 0.003 | 0.100 |
| Apple Flavour Maxarome Ac 1632 | 0.003 | 0.100 |
| TOTAL | 100.000 | 100.000 |

### Example 2

Topper (Gravy) for Dogs—

Gravy topper, XDG version 2 and version 3, see Table 3. The difference between two formulations is a higher viscosity in version 3 (XDG3).

Problem: Dry food historically has lacked palatability for dogs. To maintain a healthy physique and internal equilibrium, a tasty and vitamin-rich “meal topper” can be poured over the dry food. These gravies can come with different flavouring that targets a specific health improvement such as joint and bone, shiny coat, etc.

Delivery: Gravy/Puree

Properties: Prebiotic formula (oligosaccharides); Rich, meaty flavour with a thicker consistency; Target Audience: Offers a more diversified diet and a meal of pleasure for all dogs. Recreational usage and also to enhance the quality of the less healthy food; Older dogs who have trouble drinking enough water

Benefits and Uses: Palatable flavour for dogs, delivering nutrients and vitamins through an isotonic delivery system; and Ready to use—no preparation required before serving, just pour on top of standard dry food.

TABLE 3
The ingredients of a preferred embodiment for
a topper formulation, XDG version 2 and version 3.
Gravy Topper XDG

| | XDG V2 | XDG V3 |
|---|---|---|
| Ingredients | % | % |
| Demineralized Water | 95.04 | 94.84 |
| Glucose Anhydrous | 1.60 | 1.60 |
| Sodium Chloride | 0.22 | 0.22 |
| Potassium Chloride | 0.15 | 0.15 |
| Glycine | 0.15 | 0.15 |
| Xanthan Gum | 0.20 | 0.40 |
| Monosodium Phosphate | 0.10 | 0.10 |
| Liquid Liver Hydrolisate | 2.00 | 2.00 |
| Oligosaccharide | 0.10 | 0.10 |
| L-Glutamic Acid | 0.04 | 0.04 |
| Monosodium Glutamate | 0.19 | 0.19 |
| Burnt Sugar | 0.20 | 0.20 |
| Rebaudioside (A Steviol Glycoside A | 0.01 | 0.01 |
| 98% Min. | | |
| Total | 100.00 | 100.00 |

Preparation: premix all dries before addition to water; pH 3.8-4.2 at 20 degrees C.

### Example 3

Ready-to-Use Liquid for Dogs:

X-D Liver hydrosylate version 3 (see Table 4).

Problem: Most dogs do not prefer to drink tap water that contains chlorine. Both chlorine and ammonia are products that not only give off strong odors that dogs hate, but are also highly harmful to them; see https://www.animalwised.com/top-10-smells-dogs-hate-1568.html; incorporated herein as reference. Additionally, there does not exist any hydration liquid in the pet retail market aimed at providing nutrients and electrolyte replenishment to cats. The industry is hyper-focused on food products, leaving a huge missed opportunity for liquids. This RTU liquid can only be compared to Oralade (sold only in Europe), from which this formula is derived.

Delivery: Liquid

Properties: Fast hydration with an isotonic delivery system; Ready-to-use and palatable oral hydration liquid; Pleasant chicken aroma and taste; and Prebiotic formula (oligosaccharides)

Audience: All dogs, especially those who have been exercising, working or are recovering from illness. Benefits and Uses: Ready to use formula with no mixing required; Provides fast and efficient hydration while promoting gut health through simple amino acids and prebiotics with an isotonic delivery system; Recommended for pets requiring rehydration due to recreational activities: exercise, travel, hot weather; and Convenient when on-the-go; Packaging Sizes: Could have a variety of these.

TABLE 4
The ingredients of a preferred embodiment
for a topper formulation, XDG version 2 and version 3.
Px D Liquid Liver Hydrolisate (Version 3) (XDV3)

| Ingredients | % |
|---|---|
| Demineralized Water | 95.32 |
| Glucose Anhydrous | 1.60 |
| Sodium Chloride | 0.22 |
| Potassium Chloride | 0.15 |
| Glycine | 0.15 |
| Monosodium Phosphate | 0.10 |
| Xanthan Gum | 0.12 |
| Mercey C14028 Liver Hydrolisate | 2.00 |
| Oligosaccharide | 0.10 |
| L-Glutamic Acid | 0.04 |
| Monosodium Glutamate | 0.19 |
| Rebaudioside A 98% Min | 0.01 |
| Total | 100.00 |

### Example 4

Powder for Dogs:

X54 spray dried liver hydrolisate version 2, see Table 5.

Problem: Inconvenient and heavy water bottles make it more difficult to take your dog to the outdoors and for recreational exercise. These lightweight single servings make it easy to deliver electrolytes and rehydration to your dog.

Delivery: Powder

Properties: Fast hydration with an isotonic delivery system; Ready-to-use and palatable oral hydration powder; Pleasant chicken aroma and taste; and Prebiotic formula (oligosaccharides)

Benefits and Uses: Mixes readily with water, light shaking required; Very portable and lightweight: great for traveling, hiking, camping, exercise; Provides fast and efficient hydration while promoting gut health through simple amino acids and prebiotics with an isotonic delivery system; Recommended for pets requiring rehydration due to recreational activities: exercise, travel, hot weather; and Extremely long shelf life (longer than the Gel and RTU Liquid)

Packaging Sizes: Single serving (mix with 500 mL of water), similar to Crystal Light.

How to Use: Mix the powder with 500 mL of water and pour into an empty bowl.

TABLE 5
The ingredients of a preferred embodiment
for a powder for dogs formulation, X54 Spray
Dried Liver Hydrolisate Version 2.

| | Liquid | Powder |
|---|---|---|
| Ingredients | % | % |
| Demineralized water | 96.50 | |
| Glucose anhydrous | 1.82 | 52.00 |
| Sodium chloride | 0.22 | 6.286 |
| Potassium chloride | 0.15 | 4.286 |
| Glycine | 0.15 | 4.286 |
| Monosodium phosphate | 0.10 | 2.856 |
| Citric acid anhydrous | 0.22 | 6.286 |
| Spray dried liver hydrolisate | 0.50 | 14.286 |
| Oligosaccharide | 0.10 | 2.857 |
| L-glutamic acid | 0.04 | 1.143 |
| Monosodium glutamate | 0.19 | 5.428 |
| Rebaudioside a 98% min. | 0.01 | 0.286 |
| Total | 100.000 | 100.000 |

### Example 5

Gel for Dogs:

firm gel carrageenan-LBG, (LBG—locust bean gum), see Table 6.

Problem: Providing water to your dog on-the-go requires a portable water bowl, which is inconvenient to carry and use. This palatable hydration gel can be administered on surfaces like grass and sidewalk and require no additional equipment.

Delivery: Gel

Properties: Using an isotonic delivery system (which occurs when the gel reaches the dog's stomach), the gel provides electrolytes and hydration to your dog when they need it most; Provides fast and efficient hydration while promoting gut health through simple amino acids and prebiotics (oligosaccharides) with an isotonic delivery system; and pleasant chicken liver aroma and taste.

Benefits: When walking your dog, no water bowl is required. The gel can be administered on any surface (grass, sidewalk, etc.); Great for recreational exercising, especially in hot weather climates; Stable in a variety of temperature conditions; and Easy to carry and convenient.

Packaging Sizes: Foil bag (250 mL), single serving, or peel-wrapped cubes.

How to Use: Squeeze foil bag onto any surface (grass, sidewalk, etc.). The number of cubes given would depend on the size of the dog.

TABLE 6
The ingredients of a preferred embodiment for an edible
gel for dogs formulation, Prototype Firm Gel Carrageenan-LBG.
Preparation for gel: Premix all dries before addition to water
Disperse and dissolve in water at max 35 degrees C.
Fill into pouches, double seal, immerse for 30 minutes minimum
in boiling water. Remove from boiling water, allow to cool.
Additional non-limiting embodiment of gel consists agar and
pectin as alternatives to Carrageenan-LBG gelling composition.
Prototype Firm Gel Carrageenan-LBG

| Ingredients | % |
|---|---|
| Demineralized Water | 95.45 |
| Glucose Anhydrous | 1.69 |
| Sodium Chloride | 0.22 |
| Potassium Chloride | 0.15 |
| Glycine | 0.15 |
| Kappa Carrageenan Gum | 0.60 |
| Locust Bean Gum | 0.60 |
| Xanthan Gum | 0.20 |
| Oligosaccharide | 0.10 |
| Spray Dried Chicken Liver | 0.50 |
| L-Glutamic Acid | 0.04 |
| Monosodium Glutamate | 0.19 |
| Burnt Sugar Syrup | 0.10 |
| Rebaudioside A Min 98% | 0.01 |
| Total | 100.00 |

### Example 6

Ready-to-use liquid for cats: Isotonic liquid for cats, see Table 7.

Problem: Cats are notoriously picky about drinking water. Electrophysiological recordings show that water is not tasteless to cats; see Bartoshuk, L. M., M. A. Harned, and L. H. Parks. “Taste of water in the cat: effects on sucrose preference.” Science 171.3972 (1971): 699-701. disclosed herein as a reference. However, there does not exist any hydration liquid in the pet retail market aimed at providing nutrients and electrolyte replenishment to cats. The industry is hyper-focused on food products, leaving a huge missed opportunity for liquids. This RTU liquid can only be compared to Oralade (sold only in Europe), from which this formula is derived.

Delivery: Liquid

Properties: Fast hydration with an isotonic delivery system; Ready-to-use and palatable oral hydration liquid; Pleasant chicken or fish aroma and taste; and Prebiotic formula (oligosaccharides).

Benefits and Uses: Ready to use formula with no mixing required; Provides fast and efficient hydration while promoting gut health through simple amino acids and prebiotics with an isotonic delivery system; recommended for pets requiring rehydration due to recreational activities: exercise, travel, hot weather; and Convenient when on-the-go.

Packaging Sizes: Could have a variety of sizes.

How to Use: Pour the contents into an empty bowl.

TABLE 7
The ingredients of a preferred embodiment for an isotonic
drink for cats' formulation, Isotonic Liquid for Cats Version 1.
Isotonic Liquid for Cats (Version 1)

| Ingredients | % |
|---|---|
| Demineralized Water | 94.20 |
| Glucose Anhydrous | 1.50 |
| Sodium Chloride | 0.15 |
| Potassium Chloride | 0.10 |
| Glycine | 0.30 |
| Monosodium Phosphate | 0.10 |
| Xanthan Gum | 0.12 |
| Liquid Liver Hydrolysate | 3.00 |
| Fructo-Oligosaccharide | 0.10 |
| L-Glutamic Acid | 0.04 |
| Monosodium Glutamate | 0.19 |
| Taurine | 0.08 |
| Liquid Roast Chicken Flavour | 0.12 |
| Total | 100.00 |

Target pH = 3.3-3.6 At 20° c.

### Example 7

Powder for cats: Isotonic edible powder mix for cats, see Table 8.

Problem: It is important to keep your cat hydrated, particularly in high temperature conditions. Particularly if they spend time in the outdoors and in direct sunlight, combined with their meager daily intake of water, an electrolyte replenishment formula can be a critical tool to increase hydration. This is also a good product for older cats who have trouble drinking water or critical care patients who just underwent some medical procedure.

Delivery: Powder

Properties: Fast hydration with an isotonic delivery system; Ready-to-use and palatable oral hydration powder; Pleasant chicken of beef aroma and taste; and Prebiotic formula (oligosaccharides).

Audience: Older cats that have trouble drinking, post-trauma, but also for recreational usage.

Benefits and Uses: Mixes readily with water, light shaking required; Very portable and lightweight; Provides fast and efficient hydration while promoting gut health through simple amino acids and prebiotics with an isotonic delivery system; and Extremely long shelf life (longer than the Gel and RTU Liquid)

Packaging Sizes: Single serving (mix with 100 mL of water)

How to Use: Mix the powder with 100 mL of water and pour into an empty bowl.

TABLE 8
The ingredients of a preferred embodiment for an isotonic
powder for cats formulation, Isotonic Powder Mix For Cats Version 1.
Isotonic Powder Mix for Cats (Version 1)

| | % | % |
|---|---|---|
| Ingredients | Liquid | Powder |
| Demineralized Water | 96.00 | |
| Glucose Anhydrous | 1.34 | 33.50 |
| Sodium Chloride | 0.20 | 5.00 |
| Potassium Chloride | 0.15 | 3.75 |
| Glycine | 0.30 | 7.50 |
| Monosodium Phosphate | 0.10 | 2.50 |
| Citric Acid Anhydrous | 0.30 | 7.50 |
| Spray Dried Liver Hydrolysate | 1.00 | 25.00 |
| Fructo-Oligosaccharide | 0.10 | 2.50 |
| L-Glutamic Acid | 0.04 | 1.00 |
| Monosodium Glutamate | 0.19 | 4.75 |
| Taurine | 0.08 | 2.00 |
| Roast Chicken Flavour Powder | 0.20 | 5.00 |
| Total | 100.00 | 100.00 |

### Example 8

The oral composition for cats was prepared, according to the composition provided in Table 1 below. The dry ingredients were premixed and added to liquid ingredients. The solution was then heated to 95° C., after which the product was filled into Polyethylene terephthalate (PET) bottles at a temperature of at least 85° C. and held for 5 minutes to ensure pasteurization.

A palatability test for examining the effect of the composition of Table 1 on hydration in cats was carried out. These tests were carried out over a period of three days, with a cohort of 20 cats. The average weight of the cats was 4.5 kg. The cats were individually housed during feeding time. For the rest of the day, the cats were in groups in the playground. Each cat was offered two bowls, one containing 250 gr of plain water, and the second containing 250 gr of a composition of Table 1. There were two elements to the test: (1) initial palatability (“first choice”) and (2) total quantity consumed. The first choice of each cat was recorded on each day. The quantity consumed from each of the two bowls was measured on each day. The trial was a cross-over design, which means that on the second and third days, the contents of each bowl were replaced for each cat, and positions of each of the two bowls was switched for each cat. This is normal procedure for palatability and consumption trials.

In addition, stool consistency was evaluated, using the following grading system: 1=liquid-watery; 2=liquid-gel; 3=semi-formed; 4=formed; 5=hard-dry

A non-limiting example for oral compositions for cats is shown in Table 9:

TABLE 9
An example of oral compositions for cats:

| Ingredient | % (w/w) |
|---|---|
| Demineralized water | 90.7-97.84 |
| Glucose anhydrous | 0.9-1.55 |
| Sodium chloride | 0.1-0.2 |
| Glycine | 0.15-0.4 |
| Potassium chloride | 0.1-0.2 |
| Monosodium phosphate | 0.05-0.5 |
| Xanthan gum | 0.1-0.3 |
| Liquid liver hydrolysate | 0.5-4.0 |
| Fructo-oligosaccharide | 0.05-0.15 |
| L-Glutamic acid | 0.01-0.4 |
| Monosodium glutamate | 0.05-0.8 |
| Taurine | 0.05-0.5 |
| Meat/chicken flavor | 0.1-0.3 |
| Total | 100.00 |

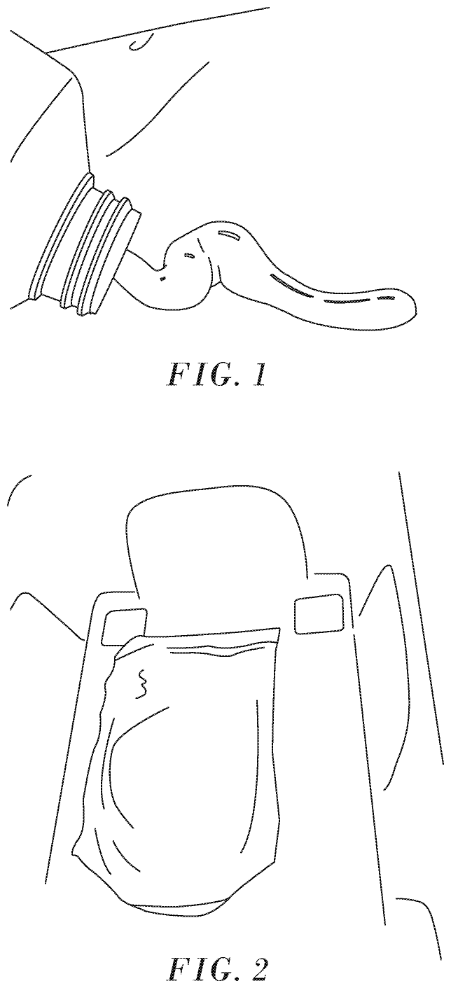

Results:
(1) Initial Palatability: As shown in FIG. 2, 69% of cats favored the composition of the present application as their first choice, compared to 31% of the cats favoring water as their first choice. This data shows a clear preference of cats for the composition of the present application, relative to water.
(2) Total quantity consumed: as mentioned above, the consumption from each of the two bowls was measured on each day, and the results shown in FIG. 1. Average intake of the composition of Table 1 was 155 gr/day/cat, which was three times the average weight of water consumed, 51 gr/day/cat. This represents a 3-fold increase in intake of the composition of the present application relative to water, which was statistically significant (P<0.0001).

The average stool grade was found to be 4 which is ‘normal feces’. Thus, it can be concluded that the ingestion of increased liquid weight had no discernable effect on the cats' normal bowel function.

### Example 9

An oral composition for dogs was prepared, according to the composition provided in Table 2 below. The dry ingredients were premixed and added to liquid ingredients. The solution was then heated to 95° C., after which the product was filled into PET bottles at a temperature of 85° C., and held at 85° C. for at least 5 minutes to ensure pasteurization.

A palatability test for examining the effect of the composition of the present application on hydration in dogs was carried out. These tests were carried out over a period of two days, with a group of 30 dogs.

The dogs were housed in a commercial kennel setting. The average weight of the dogs was 13.45 kg (range: 7.2-21.8 kg). The trial was a cross-over design, which means that on the second day, the contents of each bowl were replaced for each dog, and positions of each of the two bowls were switched for each dog. This is normal procedure for palatability and consumption trials with dogs. There were two elements to the test, namely, (1) initial palatability (“first choice”) and (2) total weight consumed. The first choice of each dog was recorded on each day. The quantity consumed from each of the two bowls was measured on each day.

Each dog was offered two bowls, one containing 1000 gr of plain water, and the second containing 1000 gr of the composition of the present application. The first choice of each dog was recorded. The quantity consumed from each of the two bowls was measured. On the second day, the contents of each bowl were replaced, and positions of each of the two bowls was switched.

In addition, stool consistency was evaluated, using the grading system from Example 1: 1=liquid-watery; 2=liquid-gel; 3=semi-formed; 4=formed; 5=hard-dry

A non-limiting example for an oral composition for dogs is shown in Table 2:

TABLE 10
An example of an oral composition for dogs:

| Ingredient | % (w/w) |
|---|---|
| Demineralized water | 90.8-97.93 |
| Glucose anhydrous | 0.9-2.0 |
| Sodium chloride | 0.1-0.3 |
| Potassium chloride | 0.1-0.2 |
| Glycine | 0.1-0.4 |
| Monosodium phosphate | 0.05-0.3 |
| Xanthan gum | 0.1-0.3 |
| Liquid liver hydrolysate | 0.5-4.0 |
| Fructo-oligosaccharide | 0.05-0.15 |
| L-Glutamic acid | 0.01-0.4 |
| Monosodium glutamate | 0.05-0.8 |
| Rebaudioside A 98% min. | 0.01-0.05 |
| Meat/chicken flavor | 0.1-0.3 |
| Total | 100.00 |

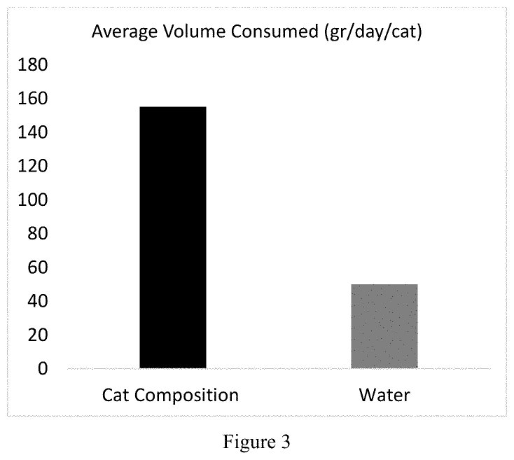

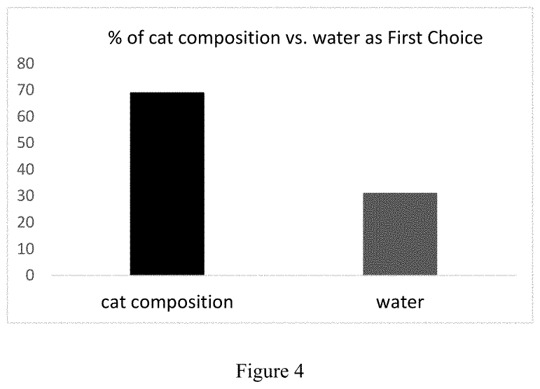

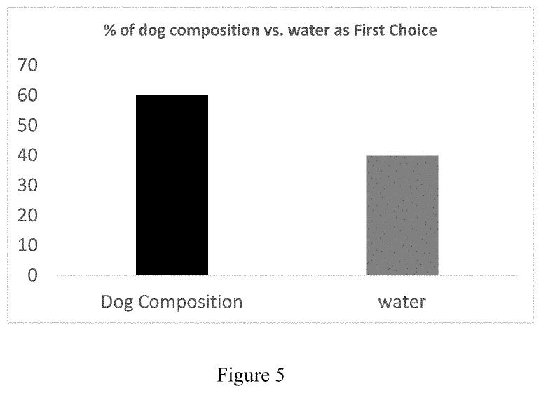

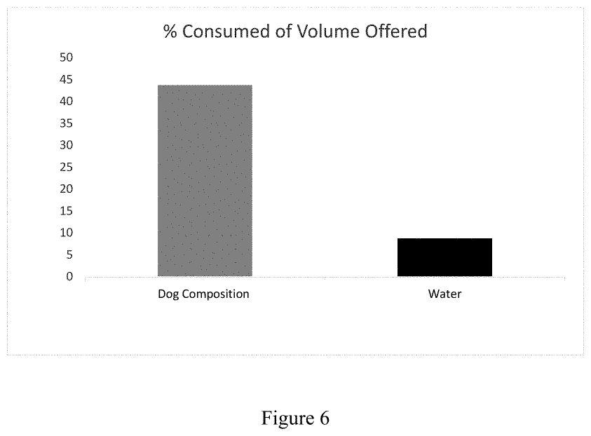

Results:
(1) Initial taste preference: As shown in FIG. 3, the majority of dogs were found to prefer the composition of Table 2, with 60% of dogs favoring the composition of Table 2, and only 40% of dogs favoring plain water as their first choice.
(2) Total consumption: As described hereinabove, the consumption from each of the two bowls was measured, and the results are shown in FIG. 4. These data show a clear and statistically significant increase in liquid intake of the composition of the present application, relative to water. The average intake of the composition of the present application was 439 gr/day/dog, compared to an average water intake of 86 gr/day/dog. Overall, during the 2 days, 44% of the composition of the present application offered (1 kg/day/dog) was consumed, compared to consumption of 9% of the weight of water offered (1 kg/day/dog). This was a significantly higher liquid intake of the composition of the present application, compared to water (P<0.0001)

The average stool grade in both treatment groups was found to be: 3-4, which was within the normal limits of semi-formed or formed, indicating that ingestion of the composition of the present application did not have any discernible effect on the dogs' normal bowel function.

All references cited herein are intended to be incorporated by reference. Although the present invention has been described above in terms of specific embodiments, it is anticipated that alterations and modifications to this invention will no doubt become apparent to those skilled in the art and may be practiced within the scope and equivalents of the appended claims. The disclosed embodiments are illustrative and not restrictive, and the invention is not to be limited to the details given herein. There are many alternative ways of implementing the invention. It is therefore intended that the disclosure and following claims be interpreted as covering all such alterations and modifications as fall within the true spirit and scope of the invention.

1. Oral isotonic compositions, characterized by no residual chlorine taste, comprising at least three members of a group consisting of steviol glycoside, citric acid monohydrate, glutamic acid, monosodium glutamate and glycine;
wherein said compositions provide hydration to mammals in need thereof.

2. The compositions of claim 1, wherein said composition is characterized as an oral isotonic drink, edible powder or edible gel.

3. The compositions of claim 2, wherein said edible gel is characterized as a solid shear thinning thixotropic gel.

4. The compositions of claim 1, wherein said composition further comprises adding at least one compound characterised as probiotics, vaccines, iron-containing compositions, minerals, salts, amino acids, medicaments, vitamins, nutraceuticals and taste enhancer.

5. The composition of claim 4, wherein said taste enhancer is selected from the group consisting of chicken flavor, fish flavor, beef flavor, apple flavor, and any combination thereof.

6. The compositions of claim 5, wherein said compositions are tailored to suit the preferences of different species or individuals.

7. The compositions of claim 1, additionally comprising at least one animal product, selected from a group consisting of meat, fish or chicken hydrolysate and meat or chicken flavorings.

8. A method for the hydration of mammals, comprising a step of administering an effective dose of a composition characterized by no residual chlorine taste, comprising at least three compounds selected from a group consisting of steviol glycoside, citric acid monohydrate, glutamic acid, monosodium glutamate and glycine.

9. The method of claim 8, wherein said composition is characterized as a-solution, oral isotonic drink, edible powder or edible gel.

10. The method according to claim 9, wherein said edible gel is characterized as a solid shear thinning thixotropic gel.

11. The method of claim 10, wherein said composition further comprises at least one compound characterised as probiotics, vaccines, iron-containing compositions, minerals, salts, amino acids, medicaments, vitamins, a taste enhancer and nutraceuticals.

12. The method of claim 11, wherein said taste enhancer is selected from the group consisting of chicken flavor, fish flavor, beef flavor, apple flavor, and any combination thereof.

13. The method of claim 12, wherein said compositions are tailored to suit the preferences of different species or individuals.

14. A method of producing a composition for inducing an animal to increase its water intake comprising steps of mixing a meat or chicken hydrolysate, meat or chicken flavorings, water, at least one sweetener, and at least one component selected steviol glycoside, citric acid monohydrate, glutamic acid, monosodium glutamate and glycine.

15. The method of claim 14, additionally comprises a step of adding at least one compound selected from a group consisting of prebiotics, vaccines, iron-containing compositions, minerals, salts, amino acids, medicaments, vitamins, taste enhancers and nutraceuticals.

16. The method of claim 15, additionally comprises a step of adding at least one compound characterised as a taste enhancer.

17. The method of claim 16, wherein said taste enhancer is selected from the group consisting of chicken flavor, fish flavor, beef flavor, apple flavor, and any combination thereof.

18. The method of claim 17, wherein said compositions are tailored to suit the preferences of different species or individuals.

19. The method of claim 14, wherein said composition is characterized as an oral isotonic drink, a solution, an edible powder or an edible gel

20. The method according to claim 19, wherein said edible gel is characterized as a solid shear thinning thixotropic gel.
